(19) United States
(12) Patent Application Publication
(10) Pub. No.: US 2025/0241529 A1
(43) Pub. Date: Jul. 31, 2025

(54) IMPROVED SYSTEMS AND METHODS FOR TESTING PERIPHERAL VISION

(71) Applicant: PERIPHEREX, INC., Menlo Park, CA (US)

(72) Inventors: Yitzchak KEMPINSKI, Geva Binyamin (IL); Jeffrey GOLDBERG, Menlo Park, CA (US)

(21) Appl. No.: 18/853,968

(22) PCT Filed: Mar. 31, 2023
(86) PCT No.: PCT/US2023/017099

§ 371 Date: Oct. 3, 2024

Related U.S. Application Data
(60) Provisional application No. 63/327,388, filed on Apr. 5, 2022.

(51) Int. Cl. A61B 3/024; A61B 3/00; A61B 3/11

(52) U.S. Cl. CPC A61B 3/024; A61B 3/0033; A61B 3/112; A61B 2560/0223

(57) ABSTRACT

Systems and methods according to present principles use touchscreen-based devices such as tablet computers or other computers incorporating touchscreens to both run the test and to receive input/output. It will be understood that any such device may be employed, so long as a display, means for user input, and means for eye tracking, are provided, and so long as its display screen is large enough to effectively test visual field.

### CROSS-REFERENCE TO RELATED APPLICATIONS

This application claims benefit of priority of U.S. Provisional Patent Application Ser. No. 63/327,388, filed Apr. 5, 2023, the contents of which are incorporated herein by reference.

### BACKGROUND

Peripheral vision, as opposed to central vision, relates to the ability to see objects and movement on the side extremes of one's vision, i.e., not in the direct line of sight.

The peripheral retina of the eyes is generally responsible for peripheral vision. The loss of peripheral vision is termed “tunnel vision”, and can be associated with glaucoma or other deteriorations of the retina or optic nerve. Thus, one potential test for glaucoma involves testing peripheral vision, and such has been implemented by perimetry tests, i.e., visual field testing.

However, such are generally associated with a complicated piece of capital equipment called a “Perimeter”, which is generally bulky and not suited for portable use. Portable and inexpensive equipment are generally highly desired as trends towards value and outcome based medicine continue.

### SUMMARY OF THE INVENTION

In an implementation, systems and methods according to present principles use touchscreen-based devices such as tablet computers or other computers incorporating touchscreens to both run the test and to receive input/output. It will be understood that any such device may be employed, so long as a display, means for user input, and means for eye tracking, are provided, and so long as its display screen is large enough to effectively test visual field.

In one aspect, the invention is directed towards a method of testing the visual field of a user, including: displaying a still element on a touch screen user interface; detecting if a user's eye or eyes are fixated on the still element; if the user's eye or eyes are not fixated on the still element, displaying an instruction to the user to look at the still element; if the user's eye or eyes are fixated on the still element, and during the time that the user's eye or eyes are fixated on the still element, displaying a flash element at a location in a peripheral vision at a first given time; receiving a signal indicating that a user saw the flash element while the user's eye or eyes were fixated on the still element; repeating the displaying a flash element at the same or another location in the peripheral vision and the receiving a signal for one or more subsequent given times; determining an indication of the visual field of the user based on the received signals.

Implementations of the invention may include one or more of the following. The receiving a signal may include receiving a tap or click indication from the user interface, or receiving an auditory indication from the user interface. The method may further include determining whether the receiving a signal occurred within a first duration following the first given time, and the determining an indication may be further based on the determining whether the receiving a signal occurred within a first duration following the first given time. The determining an indication may include calculating a visual field. The method may further include displaying an indication of the calculated visual field on the user interface. The method may further include determining a measure associated with glaucoma based on the calculated visual field. The method may further include determining a measure associated with eye or vision or other defects or maladies based on the calculated visual field. The eye or vision defect or malady may involve the peripheral vision. The method may further include determining if an eye movement has occurred during the fixation, and if so, determining a type of eye movement detected as part or all of the movement. The method may further include basing a portion of the determining an indication on the presence and type of determined eye movements.

In another aspect, the invention is directed towards a computer readable medium, including instructions for causing a computing environment to perform the method described above. The instructions may be downloadable from an online resource.

In another aspect, the invention is directed towards a method of testing the visual field of the user, including: detecting a user input indicating that a visual field test should begin; displaying a first element on a touch screen user interface; detecting if a user's eye or eyes are looking at the first element, and determining a first time duration between the display of the first element and the detecting; repeating the displaying and detecting if a user's eyes are looking at the respective element steps for a plurality of subsequent sequential elements; based on locations of the sequential elements, and/or distances therebetween, and the determined time durations, and the determined angles of user saccades, and the determined distances of user saccades, determining an indication of a visual field of the user.

Implementations of the invention may include one or more of the following. The method may further include if the user's eye or eyes are detected to be not looking at the displayed element, displaying an instruction to the user to look at the displayed element. The detecting if the user's eye or eyes are looking at the element may include detecting if the user's eye or eyes are fixated on the element. The detecting if the user's eye or eyes are looking at the element may include detecting if the user's eye or eyes moved in a direction towards the element. The detecting if the user's eye or eyes moved in a direction towards the element may include detecting if the user's eye or eyes moved in a direction from a previous element to the subsequent element. The method may further include determining a type of eye movement detected as part or all of the movement. The method may further include excluding certain types of eye movements from the detecting if a user's eye or eyes are looking at the element step.

Implementations of the invention may include one or more of the following. The method may further include dividing the testing of the visual field over multiple user sessions. At least one of the user sessions completes one or more steps of the method one or more times to perform a more coarse evaluation of the visual field and may further include performing the repeated steps at least one additional time in a user session subsequent to a previous user session to perform a more refined evaluation of the visual field. Performing the more coarse evaluation may include determining a first intensity window defined by upper and lower intensity values specifying intensity values that the user does and does not perceive, respectively, and wherein performing the more refined evaluation includes determining a second intensity window that is narrower that the first intensity window. The more coarse evaluation and the more refined evaluation may be performed for a common portion of the visual field of the user. The different user sessions may be used to test different portions of the visual field of the user. The method may further include tracking an iris size of the user while performing the testing to identify changes in a distance of an eye of the user to the touch screen user interface; and accounting for changes in the distance of the user when determining where on the touch screen user interface the still element and the flash element are to be displayed. For users wearing glasses, the method may further include capturing an iris size of the user with and without the glasses to determine a ratio between the iris size with and without glasses and identifying changes in the distance of the eye of the user to the touch screen user interface using the ratio as a calibration factor. The method may further include tracking a size of at least one additional facial feature of the user while performing the testing and using tracked changes in the iris size and the at least one additional facial feature to identify the changes in the distance of the eye of the user to the touch screen user interface. The method may further include tracking a size of at least one additional facial feature of the user while performing the testing and using tracked changes in the iris size and the at least one additional facial feature to identify the changes in the distance of the eye of the user to the touch screen user interface. The one additional facial feature includes an interpupillary distance. The method may further include displaying the still and flash elements on the touch screen user interface while a background intensity on the touch screen user interface is established at a level no greater than that needed to minimize or eliminate a user perception of ambient light reflected from the touch screen user interface. The method may further include sliding the still element to different locations while performing the testing. The fixation element may include an indicia of progress indicating to the user an amount of the testing that remains and/or is completed. The method may further include, for a given location in the visual field of the user, determining a minimum and maximum intensity level that is visible to the user, and repeating at least some steps at the given location while displaying the still element with an intensity level that is intermediate between the maximum and minimum intensity level. The intermediate intensity level may chosen to be closer to the minimum or maximum intensity level based at least in part on a response time of the user needed to see the flash element.

In a related aspect, the invention is directed towards a computer readable medium, including instructions for causing a computing environment to perform any of the above methods. Such instructions may be downloadable from an online resource.

Advantages of certain implementations of the invention may include one or more of the following. Systems and methods according to present principles provide significantly inexpensive and convenient ways to perform visual field testing, e.g., tests of peripheral vision, for the diagnosis of eye and vision ailments including glaucoma.

### BRIEF DESCRIPTION OF THE DRAWINGS

FIG. 1 illustrates a touch screen computer implementing one embodiment of systems and methods according to present principles.

FIG. 2 illustrates a touch screen computer implementing another embodiment of systems and methods according to present principles.

FIG. 3 is a flowchart illustrating a method in which the system of FIG. 1 may be employed.

FIG. 4 is a flowchart illustrating a method in which the system of FIG. 2 may be employed.

Like reference numerals refer to like elements throughout. Elements are not to scale unless specifically noted.

### DETAILED DESCRIPTION

As noted above, systems and methods according to present principles may employ in an implementation touchscreen-based devices such as tablet computers or other computers incorporating touchscreens to both run the test and to receive input/output. It will be understood that any such device may be employed, so long as a display, means for user input, and means for eye tracking, are provided, and so long as its display screen is large enough to effectively test visual field.

Two embodiments are illustrated in the attached figures. However, given this teaching, other embodiments will also be understood.

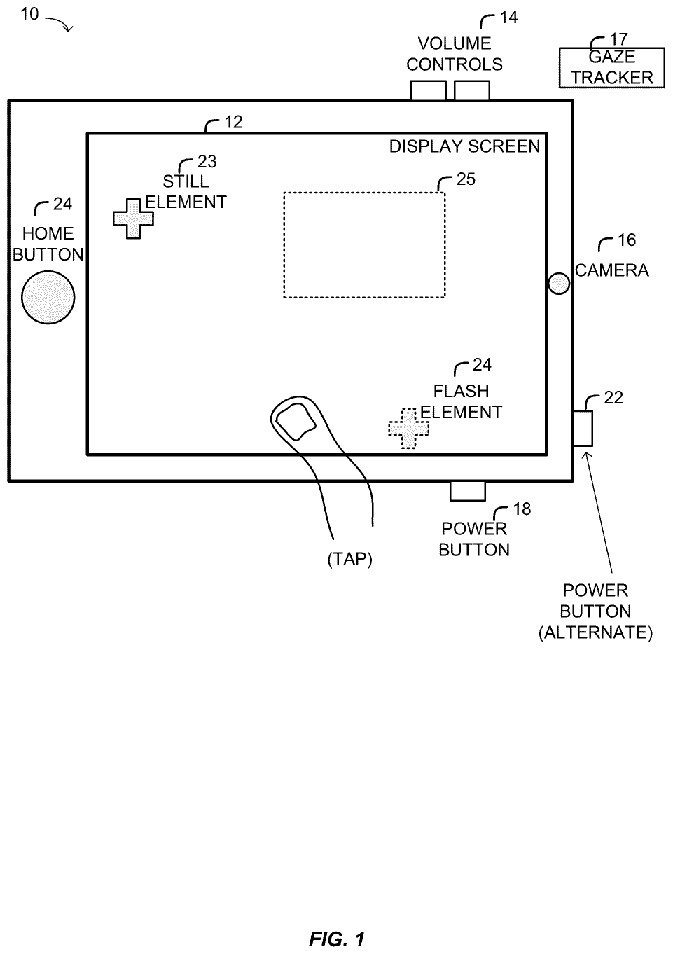

Referring first to FIG. 1, a tablet computer 10 having a display screen 12 is illustrated. The tablet computer may be, e.g., an iPad®, an Android or Windows tablet, or the like. As may be seen in the figure, volume controls 14 may be provided, as well as power button 18 or 22 or both. A camera 16 may be provided, in the same may be employed to receive a visual image of the user as well as in some implementations to implement gaze tracking by visually tracking the position of the user's eye(s), such as by use of a gaze tracker 17. A home button 24 may be provided to redirect the screen to a home page.

The display screen 12 may implement a user interface on which users may both view rendered items as well as interact with a testing application 25, e.g., by touching the touch screen UI. The testing application 25 as indicated schematically, and it will be understood that the same can cause the rendering of a still element 23, a flash element 24, as well as other elements according to implementation, as well as to receive user input, e.g., as may be detected and/or measured on the touchscreen 12.

For example, a patient may be guided with appropriate instructions on the user interface (touchscreen 12) to look at various elements on the screen and also to interact with displayed elements. For example, as shown in the figure, a still element 23 is displayed on the screen. The still element 23 is displayed in a continuous fashion. A flash element 24 may also be displayed on the screen, but the same is only displayed intermittently, e.g., as a flash. The locations of the still element and the flash element may vary, but generally, the more distant the flash element from the still element, the farther afield (from the field of view) the test is observing.

In one exemplary test, the user may be instructed to view the still element 23 and then tap the screen 12 when the flash element 24 comes into view. Other user indications of the viewer seeing the flash element may also be provided, e.g., including clicking a mouse or saying an audible word or phrase. The ability of the user to see the flash element while staring at the still element is indicative of how much peripheral vision the user possesses.

In an advanced implementation, the eye movements may be tracked, such as by the camera 16 or by other eye tracking devices and methods, and the system may wait for the eyes to fixate on the still element prior to displaying or flashing any of the flash elements. The gaze tracking or eye tracking may detect user eye fixation on the still element and may further detect, measure, or calculate user eye fixation on the flash element.

In this case, the act of fixation is not necessarily the eyes not moving at all, since if the device or head moves, the eyes generally move to compensate. Rather, here “fixation” may refer to the eyes either not moving or making slight smooth pursuits to compensate for head/device movement. The system may then wait for the user to tap the screen to confirm he or she or she is ready to start. Once the test begins, the patient fixates on the still element. As long as the patient is “fixated” on the still element, the system will present, in his or her peripheral view, flash elements 24 at various distances and angles from the still element 23.

When flash elements appear, the system tests to determine if the patient taps the screen in response, and the test may also include consideration of how much time was taken to see and respond to the flash element. If the patient looks away, e.g., a saccadic movement, from the still element, the system may stop the test and ignore the tapping of the screen. The patient may then be notified of the looking away and requested to look at the still element again to continue the test. These and other instructions to the patient may be provided by texted displayed on the screen or by audio indicators and/or notifications.

The still and flash elements may be of varying color, brightness, contrast against background, duration in time, and/or size, and may be static in any of these parameters, or varying in any of these parameters, with the goal of testing different elements of vision or variably testing or capturing the patient's attention.

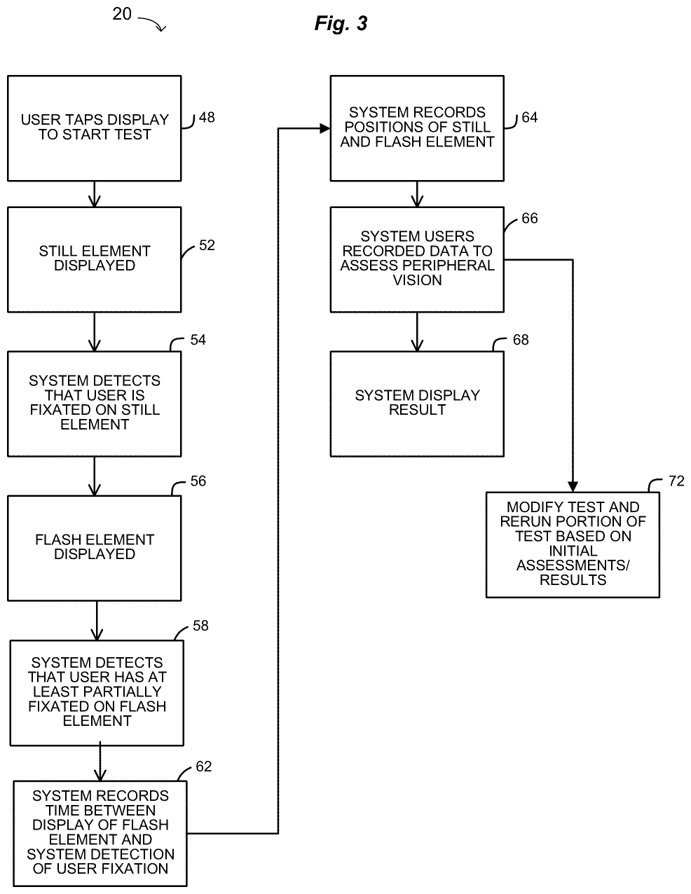

An exemplary method of the test is provided by the flowchart 20 of FIG. 3. In a first step, the user taps the display to commence the test (step 48). The still element may then be displayed (step 52). The system begins to detect whether the user has fixated on the still element, and once the system has detected such fixation, step 54 is complete and flow passes to step 56. In step 56, a flash element is displayed. The location of the flash element, as well as its duration, may be programmed or selected in a random process by the system and method.

The system may then detect that the user has at least partially fixated on the flash element (step 58). This step may be performed in various ways. For example, in one implementation, the system determines if the user has tapped on the touchscreen, and the tap is accepted as an indication that the user has seen the flash element. In another implementation, a gaze tracker or other detector may be employed to determine if the user has fixated on the flash element. Other techniques will also be understood. The system may then record the time between the display of the flash element and the detection of user fixation on the same (step 62). This step is optional, and the time may not be necessary in every assessment. Generally, however, whether a user has seen a flash element is important to record, as well as some indication of a geometric relationship between the still element and the flash element. In some cases, as peripheral vision may depend on the rotational status of a user's eyes, information about the rotational position of the user's eyes may be recorded as well, and the same used in combination with the position of the still element and the position of the flash element, as well as whether the user detected seeing the flash element, in the general assessment of user peripheral vision.

In some cases, the time notation is simply used to determine if a next element should be displayed, e.g., if a user has not seen the flash element and the test should thus continue after a predetermined period of time. In other cases, the time taken to see and respond may be used in a calculation of other aspects of the visual acuity and/or peripheral vision function of the user, e.g, response time or saccade time and direction from the still element to the flash element, or the like. For example, the faster speed and/or more accurate saccade direction with which a user reacts may be taken as evidence of better detection of the flash element and thus better peripheral vision.

As noted, the system may record positions of the still and flash elements (step 64), and finally the system may use the recorded data to provide an assessment of peripheral vision (step 66). The system may then display a result (step 68). The decline in the speed, or accuracy of the results reflected in the assessment may be taken as a reflection of a decline in the function of the visual acuity and/or peripheral vision function.

To the system and method may provide an iterative process by which a coarse assessment is made of a user's peripheral vision, e.g., using a preprogrammed set of flash elements, followed by a finer assessment. For example, an initial assessment may be the same for everyone, and may cause the user to view a predefined set of flash elements in a predetermined set of locations for a predetermined period of time. Depending on the results of this assessment, the system may branch into performing other tasks as indicated by the results of the first test, including modifying the test and rerunning portions of the test based on initial assessments and results (step 72). For example, the results of the first test may show that the user's peripheral vision is worse on the right side than on the left side. This may be confirmed in the second series of tests by rendering flash elements further out on the right and left-hand sides of the screen and determining a patient's ability to see the same.

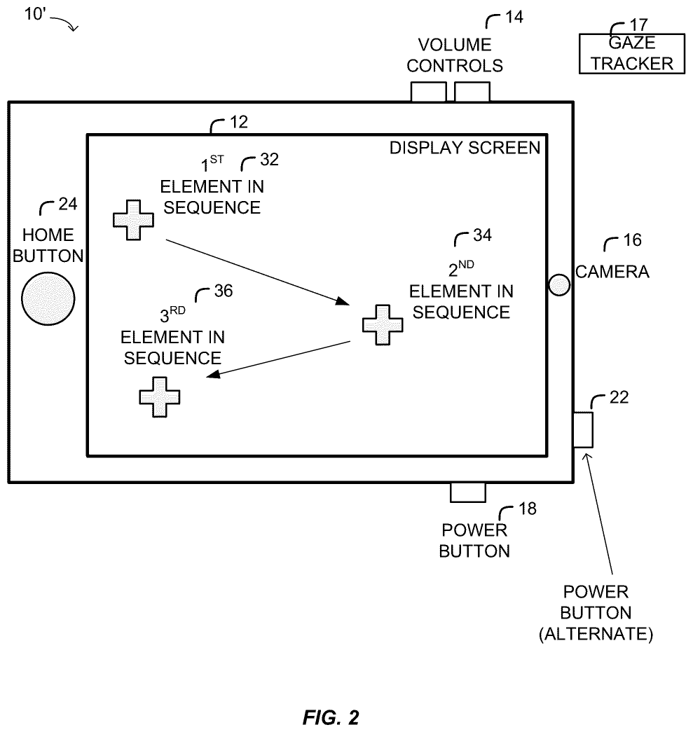

Referring next to the system 10′ of FIG. 2, a tablet computer is again employed, but a different type of test is indicated. In this approach, instead of tapping the device when a new or flash element appears, the patient is instructed and expected to change their gaze and to look at such flash elements that appear in the peripheral view. In FIG. 2, a first element in a sequence is shown as element 32, and a second element in the sequence is shown as element 34. A third element is subsequently shown as element 36. It is noted that these flash elements are displayed in a sequence.

Using a gaze tracker or in some cases also (or instead) using user tapping of the touchscreen 12, the system times the look response to the appearance of the new element to evaluate if the patient actually saw the new element and responded to its appearance. For this reason gaze tracking may be particularly useful in this implementation. In particular, the system need not measure if the user looked exactly at the new flash element, but rather if the user responded to its appearance by looking towards it. Important variables measured will include time, as again a timing element may be employed to determine how long it took for the user to respond, as well as features of the eye movement saccade including speed, direction or vector.

The still and flash elements may be of varying color, brightness, contrast against background, duration in time, and/or size, and may be static in any of these parameters, or varying in any of these parameters, with the goal of testing different elements of vision or variably testing or capturing the patient's attention. In an alternative but related implementation, the patient may be guided to always return to the same “origin” fixation point each time, or as noted the test could segue from point to point, changing brightness or size, and over this constantly moving format, calculate the visual field (see FIG. 2).

The calculations that go into the assessment of results and determination of the quality of the peripheral vision, or if determined multiple times over intervals of time, the increase or decrease in the quality of the peripheral vision, will include whether a saccade to the next point occurs, measured as “yes” or “no” where “yes” will have some limits, for example, that it must occur within 1 second and ideally within 500 milliseconds of the appearance of that next point, and that it must demonstrate a saccade in the direction of that next point, measured as degrees of deviation that must be within 30 degrees and ideally within 5 degrees of the direction of that next point, and that it must reach the distance of that next point, generally within plus or minus 25% of the full distance and ideally within 10% of the full distance, in some cases within a predetermined time period. Measures will also include if there are re-fixation movements or additional saccades to complete the fixation onto that next spot. In addition to summing these measures as “yes” or “no” for each spot, the raw data for the measures can be used to assess the quality of vision in each direction from fixation, such that measures of shorter time (e.g. closer to 200 milliseconds), closer saccade direction (e.g. closer to 0 degrees of deviation), and closer distance (e.g. exactly at the full distance), as well as a number of any needed additional re-fixation movements or saccades (e.g. 0 additional saccades needed) will be indicators of better visual function in that area of the peripheral vision.

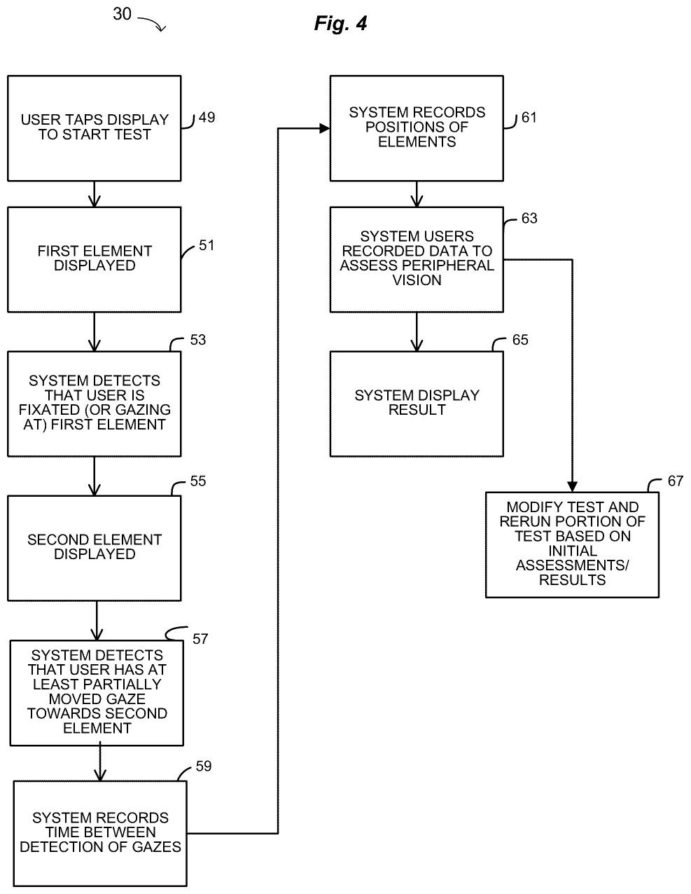

FIG. 4 depicts a flow chart 30 which may be implemented by the system of FIG. 2. As before, a user may tap the display to start the test (step 49). But in this case a first element is displayed (step 51). Unlike the method of FIG. 3, the first element may be caused to disappear upon display or rendering of a second element. However, prior to this, the system detects that the user is fixated or is otherwise gazing at the first element (step 53). Subsequently, the first element is removed and a second element is displayed (step 55). As before, the system may detect that the user has at least partially move their gaze towards the second element (step 57). The system may record the time between the detection of gazes (step 59), e.g., the time at which gaze was detected at the first element and the time in which the gaze was detected at the second element. The system records the direction and distance of the saccade towards the second element. These steps may be repeated for third, fourth, fifth, and so on, elements. The system records the positions of the elements (step 61), as well as whether the user was able to detect viewing of the elements in their peripheral vision. The system records the direction and distance of the saccade towards the subsequent elements. The system may then use for the recorded data to assess the peripheral vision of the user (step 63). The system may display the result (step 65). As before, depending on the results of prior tests, a preprogrammed test may be modified and rerun based on an initial assessment and results (step 67).

Generally, in each of these embodiments, single flash elements are displayed sequentially, although the single flash elements may be separated by a span of time in alternative implementations. The single flash elements may appear for differing amounts of time, and in some cases more than one flash element may be displayed at one time.

In one implementation, details of the ways in which eye movements can be analyzed and tracked include those disclosed in US patent application entitled “SYSTEM AND METHOD FOR ANALYSIS OF EYE MOVEMENTS USING TWO-DIMENSIONAL IMAGES”, as well as patent applications incorporated by reference therein, all of which are incorporated by reference herein. Other implementations may also be understood. In general, eye trackers may be employed which measure eye position or which measure the point of gaze. In an exemplary implementation, video-based eye trackers may be employed, in which a camera focuses on one or both eyes and records their movement. Such may also include infrared or near-infrared eye tracking techniques.

The systems and techniques described above are designed to allow the patient himself to test his visual field on general-purpose, consumer-grade equipment such as a tablet computer or the like. Unlike tests that are performed in the controlled environment of a practitioner's office on equipment that is designed and dedicated for performing such tests, the implementation of visual field testing on consumer-grade equipment and the conducting of such tests by the patient presents a number of challenges. Presented below are several examples of techniques that may be used in combination with the systems and techniques described in the aforementioned reference to address a number of these challenges. These techniques may be used individually or in any suitable combination as adjuncts to those systems and techniques described herein.

### Example 1

A primary goal of this example is to achieve a large visual field test on a small screen that may not have a sufficiently large area to test the full peripheral vision of the patient. This can be difficult to accomplish because if, for instance, the first fixed element in either of the tests described above is displayed in the center of the screen with the second element being displayed around it, the maximum possible distance between the two elements will be limited, thereby limiting how much of the patient's peripheral vision can be tested.

To address this problem, instead of using a stationary fixation element to test multiple locations on the retina, the fixation element is periodically moved to test different locations. For instance, when the fixation element is all the way to the left edge of the screen, virtually the full width of the screen is made available for testing the field of view to the right of it. Similarly, if the fixation element is moved to the bottom edge of the screen, the full height of the screen is available for testing the field of view.

Since in some cases the test generally moves the fixation element to a new location after testing a particular location on the retina, in these cases the test naturally already moves the fixation element, enabling the user room to see elements at new locations that were previously out of range. However, this may not be sufficient to test all locations in the visual field, especially since the user sometimes misses some elements due to a visual field defect. Accordingly, in order to test the full angular range of the user's peripheral vision the fixation element may be periodically moved to a new location on the screen to ensure that all visual field locations are tested and are not out of range. Importantly, while the movement of the fixation element can be a jump, more preferably the fixation element slides, which is consistent with eliciting a smooth pursuit on the part of the user, ensuring that the fixation element movement is not missed by the user. In some cases an algorithm may be used that manages which points are presented and ensures by a mixture of fixation element jumps and slides that the largest possible visual field is tested, even if the screen on which the test is presented is relatively small.

### Example 2

A primary goal of this example is to achieve a comprehensive, yet relatively short test that is able to test many different intensity levels while also achieving a high degree of discrimination between different intensity levels. This can be difficult to accomplish because the test needs to measure different levels of intensity at many different locations in the visual field, which can result in a lengthy test if all desired locations and intensity levels are tested. This may cause fatigue in the user, reducing the accuracy of the test.

To address this problem, instead of performing a single comprehensive test in a single session, the test may be divided so that it is conducted over multiple sessions. In some embodiments, instead of randomly terminating a sub-test that is performed in a single session at an arbitrary point (e.g., after a predetermined amount of time has elapsed), each sub-test may be conducted so that it provides useful information so that, for instance, the subsequent sub-test(s) can refine the previously obtained results.

For example, in some cases an initial sub-test may be performed to coarsely test the visual field at different intensity levels and different locations in the visual field and then in subsequent sub-tests more precisely determine the intensity level that the user is able to see. For example, if an initial test determines that the user can see a displayed element with an intensity level of 100 (arbitrary units) at a certain location but not an intensity level of 50, subsequent testing may be performed to determine if the user can see an intensity level of, say, 75. If so, subsequent sub-tests may be performed at that location to determine more precisely the intensity level between 50 and 75 at which the displayed element in no longer visible to the user. Alternatively, or in addition, an initial test may coarsely define those locations on the retina where a user may have visual defects and subsequent sub-tests may be performed to more precisely identify the boundary between regions where visual defects are and are not present. For instance, one sub-test(s) may broadly examine various regions of the visual field and subsequent tests can either fill in by examining locations previously unexamined or by focusing on certain areas that have been identified as possibly being more likely to produce a defect. Stated differently, in some embodiments each subsequent sub-test may iteratively refine the results obtained in a previous sub-test, improving the resolution and accuracy of the data that is ultimately obtained.

### Example 3

A primary goal of this example is to allow user to sit freely without the need for him to keep his head in a fixed position at a fixed distance to the screen while performing the test. This can be difficult to accomplish because the test is focused on testing the ability of the user to see displayed elements that are in the periphery of the user's field of vision. The locations of the displayed elements on the screen are defined by angles. The point on the screen at which the fixation elements are presented depends on the distance between the user's iris and the screen. If this distance changes from test to test, or even within a test, a displayed element that is presented at a certain pixel distance from the fixation element on the screen, will actually appear at a different location on the user's retina and thus will be testing a different, unexpected angle in the field of vision.

To address this problem, the size of the iris may be tracked, which is an average of 1.18 cm in diameter with only minimal deviations between people, regardless of ages above about 1 year old, since it is the only feature in the face that is largely consistent among people. Accordingly, the iris can be used to calculate an absolute distance to the screen, possibly along with measured changes in the sizes of other facial features. In this way the distance of the user's iris to the screen can be determined and monitored to identify any changes in the user's iris distance to the screen. Before presenting any displayed element on the screen we calculate this distance and then calculate the pixel distance on the screen necessary to present that displayed element at a certain visual angle on the screen. In this way the location of the displayed elements on the screen is constantly adapted based on the distance of the user to the screen. In some embodiments the size of the displayed elements may also be changed based on the user's iris distance to the screen. In other embodiments the calculation of visual angle changes when assembling the data for the overall visual field test. One example of a method for determining the distance between the user's iris and the screen may be found in U.S. Pat. No. 10,254,831, which is incorporated herein by reference in its entirety.

### Example 4

A primary goal of this example is to measure the distance of the user's iris to the screen even while wearing glasses. As described above the size of the iris may be used to estimate the user's distance from screen, which in turn is used to determine the distance at which the displayed elements should be presented on the screen in order to correspond to a known visual angle and therefore visual field location. If the size of the iris as detected by the camera is impacted by near-sighted or far-sighted glasses being worn by the user, this can result in erroneous calculations of the distance of the user's iris to the screen, making the user appear closer or farther from screen than he or she actually is.

To address this problem, before starting the test for the first time the camera captures an image of the iris while the user wears his glasses so that the system can calculate the user's iris size. In some embodiments this is compared to other facial features and distances that would not change with or without wearing glasses. Then the system asks the user to remove the glasses while the camera again captures an image of the iris so that the system can again calculate the user's iris size. The two iris sizes are compared and their ratio can be used as a calibration factor throughout the test to compensate for the change in size due to the glasses.

In some embodiments the comparison to other facial feature sizes and distances are used to normalize the iris size calculation, in case the patient has changed distance from the camera while adding or removing glasses. In this way the iris size used to calculate the distance between the user and the screen, which is used to translate visual angles to positions on screen throughout the test, can be calibrated to match what the distance calculation would be if the iris size of the user was captured without wearing glasses. In some cases the measurement may need to performed only a single time and the same calibration used throughout the test and possibly subsequent tests as well. In other cases the user may be prompted to periodically recalculate the calibration factor since users may sometimes wear their glasses differently, or wear different glasses, which can impact the apparent size of the iris.

In one example of the embodiment described above in which other facial sizes or distances are examined, in addition to, or instead of, measuring the iris size with and without glasses, the camera may capture the interpupillary distance, which is less impacted by glasses than the iris size. In some embodiments the interpupillary distance may be measured and used to compensate for any changes in distance that may occur between the time the user's iris is measured with glasses and measured without glasses. In some cases physician or other practitioner may enter the known interpupillary distance into the system so that the system does not need to measure the user's iris size. Rather, the interpupillary distance is captured on the screen and measured in pixels and compared to the known interpupillary distance to get a ratio and then measure the pixel iris diameter and compare to the average 1.18 cm diameter and get that ratio, The two ratios are then compared-without glasses they would be the same and therefore comparing the difference between the ratios will determine the glasses impact on the measurement. In this way knowing the pixel interpupillary distance can replace the step of removing the glasses.

As another example, a collection of distances and sizes of the head, nose, mouth, chin, ears or other features can be measured and used accordingly. This way we ensure we are only capturing the change in the apparent iris size caused by glasses and not a change in the apparent iris size that is caused by a change in the user's position.

### Example 5

A primary goal of this example is to avoid unbalanced reflections from light sources in the room off the screen, which may cause certain parts of the screen to be more difficult to see than other parts. When displayed elements are presented on the screen at varying intensity levels, the minimum level that is presented cannot go below the intensity of the background. If, for instance, the background is black, any observed reflections on the screen may prevent different areas of the screen from presenting light at lower intensity levels. If on the other hand, the whole screen is very bright, there will be fewer observed reflections but the ability to display a large range of contrasts between the brighter background and the stimulus will be more limited. Moreover, a bright screen can create reflections off the user's glasses that can block the eyes from being seen by the camera.

In general, the darker the screen the more reflections are seen from the room. On the other hand, bright light can both minimize the intensity range we have available for testing and also create reflections on the user's glasses, which can adversely impact the eye tracking and the user's ability to see the screen clearly. To address this problem the screen may be set to an intermediate intensity level (e.g. gray) which projects enough light to minimize the user's perception of light reflecting from the screen but which is not so bright that it limits the range of contrasts of the stimulus compared to background from being tested or creates reflections on the user's glasses.

It should be noted that reflections off the screen generally arise not from the overall light level in the room, but from specific light sources in the room shining onto the screen. Accordingly, in some embodiments the system's camera may detect regions of relatively high intensity light being projected towards the screen and in response present an on-screen prompt asking the user to take remedial action to reduce it. In addition (or alternatively), in some cases the user may be prompted to adjust the overall background light level in the room based on a measurement of the overall ambient light level in the room.

### Example 6

A primary goal of this example is to keep the user engaged and prevent the user's eye from “wandering” as a result of fatigue or frustration. This can be a problem because in a conventional visual field test the user's eye is fixated on one location for the duration of the test, which can be fatiguing.

To address this problem, in contrast to the conventional visual field test, in some embodiments the system described herein may move (e.g., slide) the fixation element during the test for the purpose of keeping the user more focused and engaged. This can be even more challenging if the user is anticipating the next move and may start to wander off, looking for a new fixation element if he or she is focused on the same screen location for too long. That is, if a user is fixated on the same location for more than e.g., a few seconds, due to missing displayed elements or due to wandering and a lack of focus, the fixation element can be moved (e.g., slide, to elicit smooth pursuit that is not dependent on functional peripheral vision) to a new location and the test continued from there. The movement of the fixation element can give the user a feeling of progress, reducing frustration and keeping them engaged and focused. Moreover, moving the fixation element in this manner can prevent localized screen defects (e.g., dirt or smudges on the screen, defective pixels, reflections) from causing false measurements to be obtained if these defects prevent the user from seeing a displayed element because in every new fixation location the visual field angles that surround that central fixation point are redistributed to different locations on the screen. One advantage of sliding the fixation point over simply jumping to a new location is that when sliding the user's eyes naturally slide with it (“smooth pursuit”) without losing contact with the fixation point.

### Example 7

A primary goal of this example is to allow the user to know he or she is progressing and have an understanding of how much more of the test is left. Since the test is based on the user looking at the fixation element and in some embodiments also responding and looking towards peripheral elements it is preferable not to put any additional information on the screen which may distract the user and cause the user to look at it. So it can be difficult to inform the user of the amount of progress that he or she is making in the test.

In some embodiments the fixation element itself may provide an indication of progress. For example, a progress bar, a pie graph where the filled in portion indicates the fraction of the test that is completed, or some other feature may be incorporated into the fixation element to indicate the amount of the test that has completed and/or that remains. It is also envisioned as part of this invention that the changing element(s) in the fixation element will assist with user engagement through the duration of the test.

### Example 8

A primary goal of this example is to quickly find in each location of the user's visual field the threshold intensity where the user stops seeing the displayed element. This can be a problem because there are many locations in the user's visual field to be tested and we want to find the exact threshold intensity that the user can see at each location. If we test all locations at many intensity levels it will take a very long time. In some embodiments, where frequent testing is performed, we would like to use previously obtained test data to shorten the test, but this presents problems because we need to make sure that the data is still relevant and hasn't changed.

To address this problem, an algorithm can be employed that defines visible and nonvisible intensity values (“V” and “NV”). The visible is the maximum tested value that the user could see and the nonvisible is the lowest intensity value that the user could see. In each iteration of the algorithm the system presents the middle (“M”) value between V and NV. If the M value was seen then V=M; if the M value wasn't seen then NV=M. In the next iteration, M will be halfway between the new values of V and NV, and so on. In this way the test can very quickly slide down the range between V and NV to thereby converge on the threshold intensity value. In some embodiments, instead of simply-assuming the threshold is halfway between V and NV the threshold may be assumed to be closer to V or closer to NV based in part on the user's response time needed to see a displayed element. For instance, if V=100 and NV is 50 and the user had responded very quickly to 100, we can estimate that his threshold value is closer to 50 than 100. On the other hand, if the user was slow to respond to 100 even if he or she did see it, then the threshold value is likely closer to 100 than 50.

In some embodiments, if there are V and NV intensity values available from previous tests they can be used as a starting point, but they may be designated as being “unvalidated”. What this means is that the test will not end unless either new V and NV are generated or the same “unvalidated” value is retested. For example, if V=200 and NV=100 in a previous test, these values may be used as starting points, but are defined as “unvalidated”. In the first iteration the M value may be, e.g. 150. If the user sees it, then V=150. If the test were to end here we would still have NV=100, but designated as being “unvalidated,” so the system, before ending the test, will retest at 100 to ensure its actually nonvisible before validating it. This ensures the test uses previous data to better guess the intensity range that should be tested without assuming that previous data is still valid. Note that in some cases previously obtained NV and V values that are designated as “unvalidated” may be associated with an expiration date. For example if the previous test was conducted yesterday and the expiration date has not passed, I can assume that the NV and V values are still valid, but if the previous test was conducted a month ago and the expiration date has passed, the NV and V values may be designated as “unvalidated” and therefore these values will need additional validation as described in the algorithm above.

It should be noted that each location in the peripheral field being tested has a V and NV value associated with it. However, in some embodiments the algorithm may also use the V and NV values of neighboring locations, or an average of neighboring locations, to estimate the intensity range that should be tested. Finally, it should also be noted that in some cases the user may not see a displayed element at a particular location at all, so in this case NV=maximum intensity and the V is not relevant.

The various embodiments of systems and methods described above may be fully implemented in any number of computing devices. Typically, instructions are laid out on computer readable media, generally non-transitory, and these instructions are sufficient to allow a processor in the computing device to implement the method of the invention. The computer readable medium may be a hard drive or solid-state storage having instructions that, when run, are loaded into random access memory. Inputs to the application, e.g., from the plurality of users or from any one user, may be by any number of appropriate computer input devices. For example, users may employ a keyboard, mouse, touchscreen, joystick, trackpad, other pointing device, or any other such computer input device to input data relevant to the calculations. Data may also be input by way of an inserted memory chip, hard drive, flash drives, flash memory, optical media, magnetic media, or any other type of file-storing medium. The outputs may be delivered to a user by way of a video graphics card or integrated graphics chipset coupled to a display that maybe seen by a user. Alternatively, a printer may be employed to output hard copies of the results. Given this teaching, any number of other tangible outputs will also be understood to be contemplated by the invention. For example, outputs may be stored on a memory chip, hard drive, flash drives, flash memory, optical media, magnetic media, or any other type of output. It should also be noted that the invention may be implemented on any number of different types of computing devices, e.g., personal computers, laptop computers, notebook computers, net book computers, handheld computers, personal digital assistants, mobile phones, smart phones, tablet computers, and also on devices specifically designed for these purpose. In one implementation, a user of a smartphone or WiFi-connected device downloads a copy of the application to their device from a server using a wireless Internet connection. An appropriate authentication procedure and secure transaction process may provide for payment to be made to the seller. The application may download over the mobile connection, or over the WiFi or other wireless network connection. The application may then be run by the user. Such a networked system may provide a suitable computing environment for an implementation in which a plurality of users provide separate inputs to the system and method. In the below system where visual field testing is contemplated, the plural inputs may allow plural users to input relevant data at the same time.

1. A method of testing the visual field of a user, comprising:
a. displaying a still element on a touch screen user interface;
b. detecting if a user's eye or eyes are fixated on the still element;
c. if the user's eye or eyes are not fixated on the still element, displaying an instruction to the user to look at the still element;
d. if the user's eye or eyes are fixated on the still element, and during the time that the user's eye or eyes are fixated on the still element, displaying a flash element at a first location in a peripheral vision at a first given time;
e. receiving a signal indicating that a user saw the flash element while the user's eye or eyes were fixated on the still element;
f. repeating the displaying a flash element at the first or another location in the peripheral vision and the receiving a signal for one or more subsequent given times;
g. determining an indication of the visual field of the user based on the received signals; and
h. repeating steps a-f one or more times while displaying the still element at different locations on the touch screen user interface to thereby enhance an amount of the peripheral vision that is tested.

2. The method of claim 1, wherein at least one of the times steps a-f is repeated, displaying the still element at an edge of the touch screen user interface.

3. The method of claim 1, wherein the repeating of steps a-f one or more times while displaying the still element at different locations on the touch screen user interface is performed a sufficient number of times to test all locations in the peripheral field of the user.

4. The method of claim 1, wherein the repeating of steps a-f one or more times while displaying the still element at different locations on the touch screen user interface includes sliding the still element to the different locations.

5. The method of claim 1, further comprising dividing the testing of the visual field over multiple user sessions.

6. The method of claim 5, wherein at least one of the user sessions completes steps a-f one or more times to perform a more coarse evaluation of the visual field and further comprising performing steps a-f at least one additional time in a user session subsequent to the at least one user session to perform a more refined evaluation of the visual field.

7. The method of claim 6, wherein performing the more coarse evaluation includes determining a first intensity window defined by upper and lower intensity values specifying intensity values that the user does and does not perceive, respectively, and wherein performing the more refined evaluation includes determining a second intensity window that is narrower that the first intensity window.

8. The method of claim 7, wherein the more coarse evaluation and the more refined evaluation are performed for a common portion of the visual field of the user.

9. The method of claim 5, wherein different user sessions are used to test different portions of the visual field of the user.

10. The method of claim 1, further comprising: tracking an iris size of the user while performing the testing to identify changes in a distance of an eye of the user to the touch screen user interface; and accounting for changes in the distance of the user when determining where on the touch screen user interface the still element and the flash element are to be displayed.

11. The method of claim 10, further comprising, for users wearing glasses:
capturing an iris size of the user with and without the glasses to determine a ratio between the iris size with and without glasses; and
identifying changes in the distance of the eye of the user to the touch screen user interface using the ratio as a calibration factor.

12. The method of claim 10, further comprising tracking a size of at least one additional facial feature of the user while performing the testing and using tracked changes in the iris size and the at least one additional facial feature to identify the changes in the distance of the eye of the user to the touch screen user interface.

13. The method of claim 11, further comprising tracking a size of at least one additional facial feature of the user while performing the testing and using tracked changes in the iris size and the at least one additional facial feature to identify the changes in the distance of the eye of the user to the touch screen user interface

14. The method of claim 13, wherein the one additional facial feature includes an interpupillary distance.

15. The method of claim 1, further comprising displaying the still and flash elements on the touch screen user interface while a background intensity on the touch screen user interface is established at a level no greater than that needed to minimize or eliminate a user perception of ambient light reflected from the touch screen user interface.

16. The method of claim 1, further comprising sliding the still element to different locations while performing the testing.

17. The method of claim 1, wherein the repeating of steps a-f one or more times while displaying the still element at different locations on the touch screen user interface includes sliding the still element to the different locations.

18. The method of claim 1, wherein the fixation element includes an indicia of progress indicating to the user an amount of the testing that remains and/or is completed.

19. The method of claim 1, for a given location in the visual field of the user, determining a minimum and maximum intensity level that is visible to the user, and further comprising repeating steps a-f at the given location while displaying the still element with an intensity level that is intermediate between the maximum and minimum intensity level.

20. The method of claim 19, wherein the intermediate intensity level is chosen to be closer to the minimum or maximum intensity level based at least in part on a response time of the user needed to see the flash element.

21. A computer readable medium, comprising instructions for causing a computing environment to perform the method of claim 1.

22. The medium of claim 21, wherein the instructions are downloadable from an online resource.

23. A method of testing the visual field of the user, comprising:
a. detecting a user input indicating that a visual field test should begin;
b. displaying a first element on a touch screen user interface;
C. detecting if a user's eye or eyes are looking at the first element, and determining a first time duration between the display of the first element and the detecting;
d. repeating the displaying and detecting if a user's eyes are looking at the respective element steps for a plurality of subsequent sequential elements; and
e. based on locations of the sequential elements, and/or distances therebetween, and the determined time durations, and the determined angles of user saccades, and the determined distances of user saccades, determining an indication of a visual field of the user; and
f. tracking an iris size of the user while performing the testing to identify changes in a distance of an eye of the user to the touch screen user interface; and accounting for changes in the distance of the user when determining where on the touch screen user interface the still element and the flash element are to be displayed.

24. The method of claim 23, further comprising, for users wearing glasses:
capturing an iris size of the user with and without the glasses to determine a ratio between the iris size with and without glasses; and
identifying changes in the distance of the eye of the user to the touch screen user interface using the ratio as a calibration factor.

25. The method of claim 23, further comprising tracking a size of at least one additional facial feature of the user while performing the testing and using tracked changes in the iris size and the at least one additional facial feature to identify the changes in the distance of the eye of the user to the touch screen user interface.

26. The method of claim 24, further comprising tracking a size of at least one additional facial feature of the user while performing the testing and using tracked changes in the iris size and the at least one additional facial feature to identify the changes in the distance of the eye of the user to the touch screen user interface

27. The method of claim 26, wherein the one additional facial feature includes an interpupillary distance.

28. The method of claim 23, further comprising displaying the still and flash elements on the touch screen user interface while a background intensity on the touch screen user interface is established at a level no greater than that needed to minimize or eliminate a user perception of ambient light reflected from the touch screen user interface.

29. The method of claim 23, further comprising sliding the still element to different locations while performing the testing.

30. The method of claim 23, wherein the repeating of steps a-f one or more times while displaying the still element at different locations on the touch screen user interface includes sliding the still element to the different locations.

31. The method of claim 23, wherein the fixation element includes an indicia of progress indicating to the user an amount of the testing that remains and/or is completed.

32. The method of claim 23, for a given location in the visual field of the user, determining a minimum and maximum intensity level that is visible to the user, and further comprising repeating steps a-f at the given location while displaying the still element with an intensity level that is intermediate between the maximum and minimum intensity level.

33. The method of claim 32, wherein the intermediate intensity level is chosen to be closer to the minimum or maximum intensity level based at least in part on a response time of the user needed to see the flash element.

34. A computer readable medium, comprising instructions for causing a computing environment to perform the method of claim 23.

35. The medium of claim 34, wherein the instructions are downloadable from an online resource.

36. The method of claim 23, further comprising repeating steps a-d one or more times while displaying the first element at different locations on the touch screen user interface to thereby enhance an amount of the peripheral vision that is tested.

37. The method of claim 36, wherein at least one of the times steps a-d is repeated, displaying the first element at an edge of the touch screen user interface.

38. The method of claim 36, wherein the repeating of steps a-d one or more times while displaying the first element at different locations on the touch screen user interface is performed a sufficient number of times to test all locations in the peripheral field of the user.

39. The method of claim 1, wherein the repeating of steps a-f one or more times while displaying the still element at different locations on the touch screen user interface includes sliding the still element to the different locations.

40. The method of claim 23, further comprising dividing the testing of the visual field over multiple user sessions.

41. The method of claim 40, wherein at least one of the user sessions completes steps a-d one or more times to perform a more coarse evaluation of the visual field and further comprising performing steps a-f at least one additional time in a user session subsequent to the at least one user session to perform a more refined evaluation of the visual field.

42. The method of claim 41, wherein performing the more coarse evaluation includes determining a first intensity window defined by upper and lower intensity values specifying intensity values that the user does and does not perceive, respectively, and wherein performing the more refined evaluation includes determining a second intensity window that is narrower that the first intensity window.

43. The method of claim 42, wherein the more coarse evaluation and the more refined evaluation are performed for a common portion of the visual field of the user.

44. The method of claim 40, wherein different user sessions are used to test different portions of the visual field of the user.
